(19) United States
(12) Patent Application Publication
(10) Pub. No.: US 2004/0176682 A1
(43) Pub. Date: Sep. 9, 2004

(54) Method and apparatus for reducing exposure to an imaging beam

(76) Inventor: Kieran P. Murphy, Baltimore, MD (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/376,618

(22) Filed: Mar. 3, 2003

(51) Int. Cl. A61B 5/05; A61M 25/00

(52) U.S. Cl. 600/424; 600/585

(57) ABSTRACT

A method and apparatus for reducing exposure to an imaging beam is provided. In an embodiment, such an apparatus comprises a guiding catheter for insertion in the femoral artery until the tip of the guiding catheter is proximal to a blood clot in the brain of the patient. The guiding catheter can then receive a microcatheter which has an angioplasty balloon on the tip and which is passed through the guiding catheter. A graduation towards the proximal end of the microcatheter aligns with a gauge near the proximal end of the guiding catheter and thereby indicates when the microcatheter is about to exit the distal tip of the guiding catheter. The application of doses of an imaging beam can be avoided during the insertion of the microcatheter up to the point where the graduation aligns with the gauge, thereby reducing the amount of imaging beams that would be used during the procedure were the graduation and gauge to be absent.

### FIELD OF THE INVENTION

The present invention relates to generally to medical imaging and more particularly relates to a method and apparatus for reducing exposure to an imaging beam such as an X-ray or the like.

### BACKGROUND OF THE INVENTION

Microcatheters introduced through a guiding catheter via the femoral artery are well known and can be used to navigate into the patient's torso or head, and be equipped with different types of tips, according to the procedure being performed. For example, such a microcatheter may be up to one meter or even one-and-a-half meters long and may be equipped to assist in the treatment of an aneurysm in the patient's brain. Due to the length of these microcatheters, enormous skill is required on the part of the surgeon introducing the microcatheter—especially since the target area in the patient's brain may be less than five millimeters in diameter. Typically, the surgeon will blindly introduce a large portion of the microcatheter through the guiding catheter, with only a general idea of where the distal tip of the microcatheter is located at any given time within the patient. The final length of the microcatheter is then introduced under image guidance, such as using a series of X-ray pictures, to determine exactly where the distal tip of the catheter is located in relation to the target area in the patient's brain. This can expose the patient to an undesirable number of X-ray doses. Also, if the surgeon “guesses” incorrectly, it is possible that the surgeon will overshoot the target area before relying on image guidance, and thereby possibly leading to patient injury.

### SUMMARY OF THE INVENTION

It is therefore an object of the present invention to provide a novel method and apparatus for reducing exposure to an imaging beam that obviates or mitigates at least one of the above-identified disadvantages of the prior art.

In a first aspect of the invention there is provided an apparatus for reducing exposure to an imaging beam comprising a guiding catheter having a distal tip for insertion to a target area and a proximal tip. The apparatus also comprises a first microcatheter for insertion into the guiding catheter's proximal tip, the first microcatheter having a distal tip that includes a device for treating a condition corresponding to the target area. The apparatus also comprises a locating means operably associated with at least one of the guiding catheter and the first microcatheter for indicating when the distal tip of the microcathether is about exit the distal tip of the guiding catheter at a predetermined point during the insertion.

In a particular implementation of the first aspect, the first microcatheter is hollow and the device is a second microcatheter for insertion into a proximal tip of the first microcatheter and operable to exit the first microcathether's distal tip. The apparatus further includes a second locating means operably associated with the second microcatheter for indicating when the second microcathether is about to exit the distal tip of the first microcathether during insertion of the second microcatheter.

In a particular implementation of the first aspect, the second microcatheter is selected from the group consisting of a microwire and a microcoil. In a particular implementation of the first aspect the device is a stent.

In a particular implementation of the first aspect, the locating means is a graduation disposed on the first microcatheter that lines up with a predetermined location on the guiding catheter to provide a visual indication of when the distal tip of the microcatheter is about exit the distal tip of the guiding catheter. The apparatus can further include a Touhy-Borst adapter releasably-connectable to the guiding catheter. The predetermined location can be the proximal open end of the adapter.

In a particular implementation of the first aspect the locating means is a textured surfaces located on the first microcatheter that lines up with a predetermined location on the guiding catheter to provide a tactile indication of when the distal tip of the microcatheter is about exit the distal tip of the guiding catheter.

In a particular implementation of the first aspect the apparatus further includes a Touhy-Borst adapter releasably-connectable to the guiding catheter, and the locating means is bulge located on the first microcatheter that has an exterior diameter slightly smaller than an interior diameter of the guiding cathether, such that a resistance to insertion is experienced when the bulge enters a proximal end of the adapter to indicate when the distal tip of the microcatheter is about exit the distal tip of the guiding catheter.

In a particular implementation of the first aspect the locating means provides the indication when a distal tip of the microcatheter is at a distance of between about zero millimeters to about thirty millimeters from exiting the distal tip of the guiding catheter. The distance can be from between about two millimeters to about fifteen millimeters. The distance can be from between about five millimeters to about ten millimeters. The distance can be about seven millimeters.

In a second aspect of the invention there is provided an apparatus for reducing exposure to an imaging beam comprising a guiding catheter having a distal tip for insertion to a target area and a proximal tip, and an adapter releasably connectable to the guiding catheter. The adapter has a proximal opening at a first end and a connection means at the opposite end. The connection means is for connection to the guiding catheter's proximal tip. The adapter is made from a clear material and includes an gauge. The apparatus also includes a first microcatheter for insertion into the proximal opening of the guiding catheter, and through the guiding catheter. The first microcatheter has a distal tip that includes a device for treating a condition in the target area when the device exits the guiding catheter's distal tip. The first microcatheter includes a graduation located towards a proximal end of the first microcatheter, such that when the graduation is lined-up with the gauge the distal tip of the microcatheter is at a predefined distance from exiting the guiding catheter's distal tip.

In a particular implementation of the second aspect, the distance is between about zero millimeters to about thirty millimeters. The distance can be between about two millimeters to about fifteen millimeters. The distance can be between about five millimeters to about ten millimeters. In a particular implementation of the second aspect, wherein the distance is about seven millimeters.

In a third aspect of the invention there is provided a method of inserting a cathter comprising:

inserting a guiding catheter into an incision in a patient's skin and passing the guiding catheter through one or more blood vessels until a distal tip of the guiding catheter reaches a target area within the patient; and,

inserting a microcatheter into a proximal opening of the guiding catheter until a locating means located on one of the microcatheter and the guiding catheter indicates that a distal tip of the microcather is at a predetermined distance from exiting a distal tip of the guiding catheter.

In a particular implementation of the third aspect, there is provided the additional steps of:

exposing the target area to an imaging beam to determine a location of the distal tip of the microcatheter in relation to the target area;

directing the distal tip of the microcatheter towards the target area using the imaging beam for guidance;

repeating, as necessary, the exposing step and the directing step until the distal tip of the microcatheter is in a desired location in relation to the target area.

The target area can be any treatable location in a patient's body, such as a blood clot in the patient's head. The guiding catheter can be inserted into an incision at any desired or suitable location on a patient's body, such as into the a vein or an artery, such as the femoral or brachial artery or through a vertebral body of the patient.

### BRIEF DESCRIPTION OF THE DRAWINGS

The present invention will now be explained, by way of example only, with reference to certain embodiments and the attached Figures in which:

FIG. 1 is an isometric view of an apparatus for reducing exposure to an imaging beam;

FIG. 2 shows the apparatus of FIG. 1 being used by a surgeon on patient;

FIG. 3 is an exploded view of various components of the apparatus of FIG. 1;

FIG. 4 shows the apparatus in FIG. 3 with the microcatheter advanced within the guiding catheter; FIG. 5 shows the apparatus of FIG. 4 with the microcatheter tip adjacent the target area in the patient;

FIG. 6 is a partial view of an apparatus in accordance with another embodiment of the invention;

FIG. 7 is a partial view of an apparatus in accordance with another embodiment of the invention;

FIG. 8 is a partial view of an apparatus in accordance with another embodiment of the invention;

FIG. 9 is a partial view of an apparatus in accordance with another embodiment of the invention; and,

FIG. 10 is an exploded view of the apparatus shown in FIG. 8; and,

FIG. 11 is an isometric view of an apparatus for reducing exposure to an imaging beam in accordance with another embodiment of the invention.

### DETAILED DESCRIPTION OF THE INVENTION

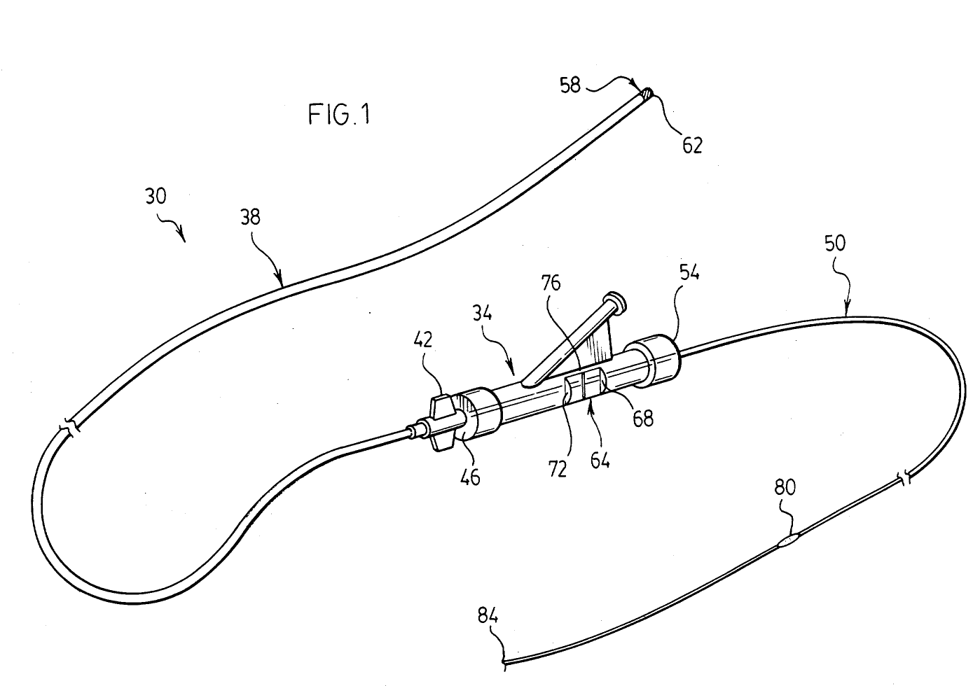

Referring now to FIGS. 1-5, an apparatus for reducing exposure to an imaging beam is indicated generally at 30. Apparatus 30 comprises a Touhy-Borst adapter 34 releasably-connectable to a guiding catheter 38 via a connector 42 (such as a luer-lock or the like) located at the distal end 46 of adapter 34. Apparatus 30 further comprises a microcathether 50 which is insertable within an open proximal end 54 of adapter 34 and into the lumen of guiding catheter 38.

Guiding catheter 38 is typically made of a flexible material such as silicon, and includes a radioopaque marker 58 at its distal tip 62 that makes the tip of marker 38 visible under an X-ray or other imaging beam under which guiding catheter 38 is intended for use.

In a presently preferred embodiment, adapter 34 is typically made from clear plastic so that microcatheter 50 is visible when being passed through adapter 34. Adapter 34 also includes a gauge 64 that occupies the circumference of a central portion of adapter 34 and is defined by a proximal indicator 68, a distal indicator 72 and a central indicator 76 located half-way between indicators 68 and 72.

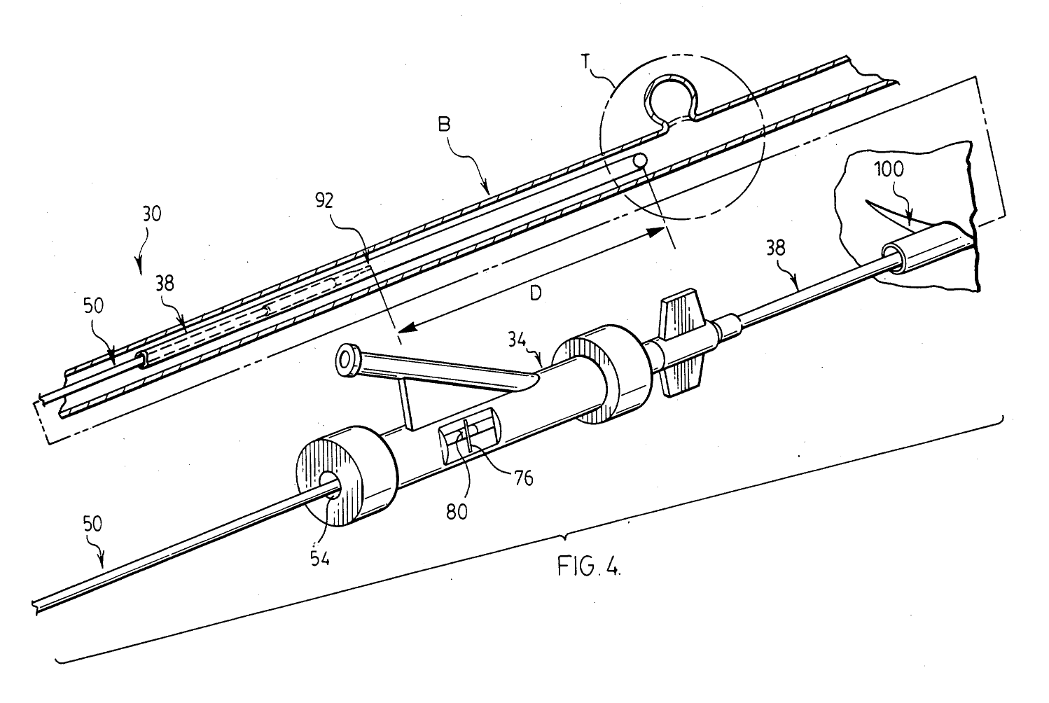

As best seen in FIG. 4, microcatheter 50 includes at least one graduation 80 located towards a proximal tip 84 of microcatheter 50, and an angioplasty balloon 88 (which includes a stent) located at the distal tip 92 of microcatheter 50. Graduation 80 is located at a position along microcatheter 50 such that when graduation 80 is aligned with central indicator 76, then tip 92 will be located at a distance (shown in FIG. 4 as “D”) of about zero millimeters to about thirty millimeters from exiting the tip 62 of guiding catheter 38. More preferably, tip 92 will be located at a distance D of about two millimeters to about fifteen millimeters from exiting the tip 62 of guiding catheter 38. Still more preferably, distance D will be about five millimeters to about ten millimeters. In a presently preferred embodiment however, when graduation 80 is aligned with central indicator 76, then tip 92 will be located at a distance D of about seven millimeters from exiting the tip 62 of guiding catheter 38.

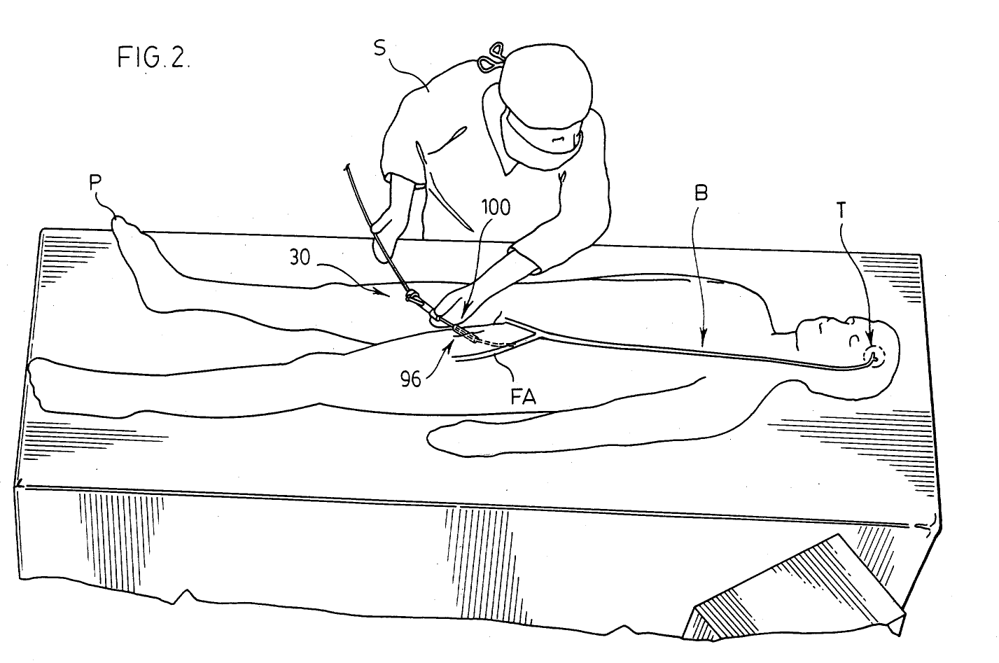

As seen in FIG. 2, a surgeon S can use apparatus 30 to treat a patient P. First, surgeon S will make an incision 96 in the thigh to provide access to the femoral artery. Next, surgeon S will place a plastic sleeve 100 in incision 96 to provide an open channel into the femoral artery FA. Surgeon S will then feed guiding catheter 38 into one or more blood vessels B towards a target area T of patient P's brain. In the present example, target area T is an blood clot, but other types and locations of target area T will occur to those of skill in the art. It is to be understood that blood vessels B as shown in FIG. 2 are simplified to represent a complex passage of arteries and blood vessels that define a pathway between incision 96 and target area T.

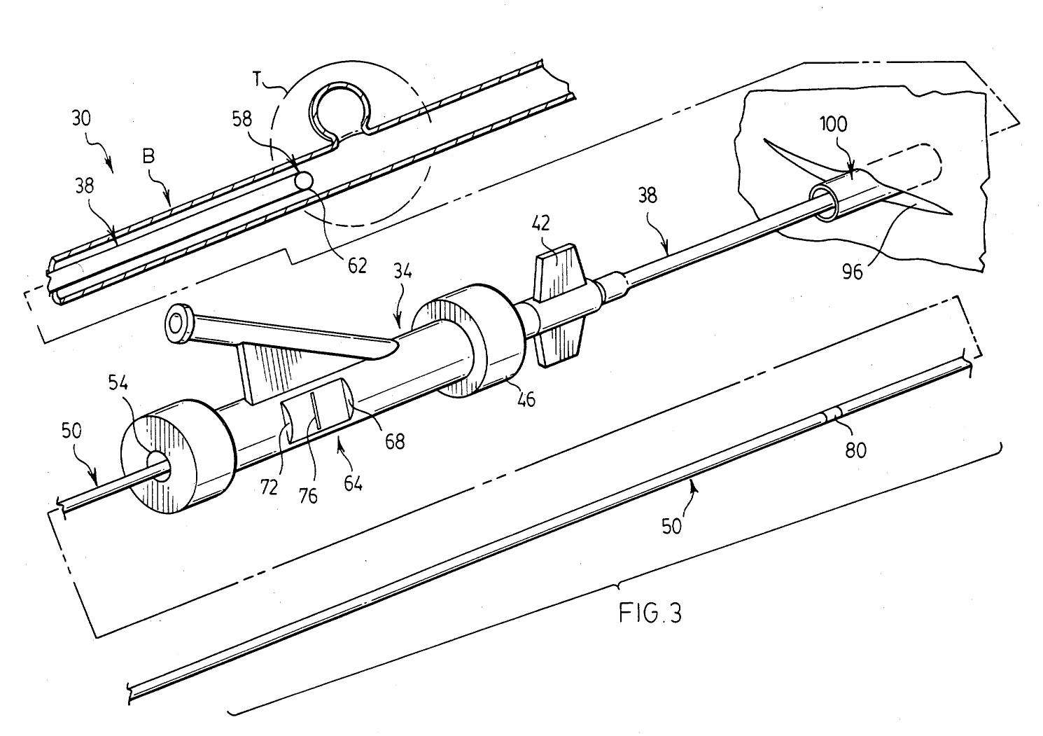

Surgeon S will typically insert guiding catheter 38 under X-ray guidance by taking an X-ray image along artery A at various intervals to determine where tip 62 is located within artery A. Recall that, since tip 62 includes radioopaque marker 58, marker 58 will be visible under X-ray beams. (Note that, in the present embodiment, the entirety of guiding catheter 38 is also radioopaque, but of a different radioopacity than marker 58, to help the surgeon S distinguish between these two parts of catheter 38. In this manner, surgeon S will continue to insert guiding catheter 38 until tip 62 is just proximal to target area T within blood vessel B, as best seen in FIG. 3.

As best seen in FIG. 3, once guiding catheter 38 is in proper location, distal tip 92 of microcatheter 50 is then inserted into proximal end 54 of adapter 34 and then fed through the lumen of guiding catheter 38 so that distal tip 92 is urged towards tip 62 of guiding catheter 38. During the insertion of microcatheter 50 into guiding catheter 38, surgeon S will watch the position of graduation 80 in relation to proximal end 54. In general, surgeon S may be able to insert microcatheter 50 at a relatively rapid rate prior to the point of graduation 80 actually entering proximal end 54 of adapter 34, and in any event, such insertion need not be performed using X-ray or other image guidance, as the location of graduation 80 is such that an indication is provided that tip 92 remains within the lumen of guiding catheter 38 as long as graduation 80 remains does not pass central indicator 76 of gauge 64.

Thus, as best seen in FIGS. 3 and 4, once graduation 80 enters proximal end 54 of adapter 34, surgeon S will use additional care as surgeon S continues to insert microcatheter 50, watching carefully as graduation 80 approaches central indicator 76, and finally ceasing further insertion once graduation 80 is actually aligned with central indicator 76. The alignment of graduation 80 with central indicator 76 is shown in FIG. 4, which also shows tip 92 located a predefined distance of about seven millimeters from exiting tip 62 of guiding catheter 38.

Surgeon S will then continue to slowly insert microcatheter 50 within guiding catheter 38, but will now rely on X-ray guidance to provide an image of where tip 92 is located in relation to target area T. As best shown in FIG. 5, such insertion under X-ray guidance thus continues until tip 92 and angioplasty balloon 88 are in a desired position in relation to target area T. Having located tip 92 in the desired location, target area T can then be treated in the usual manner.

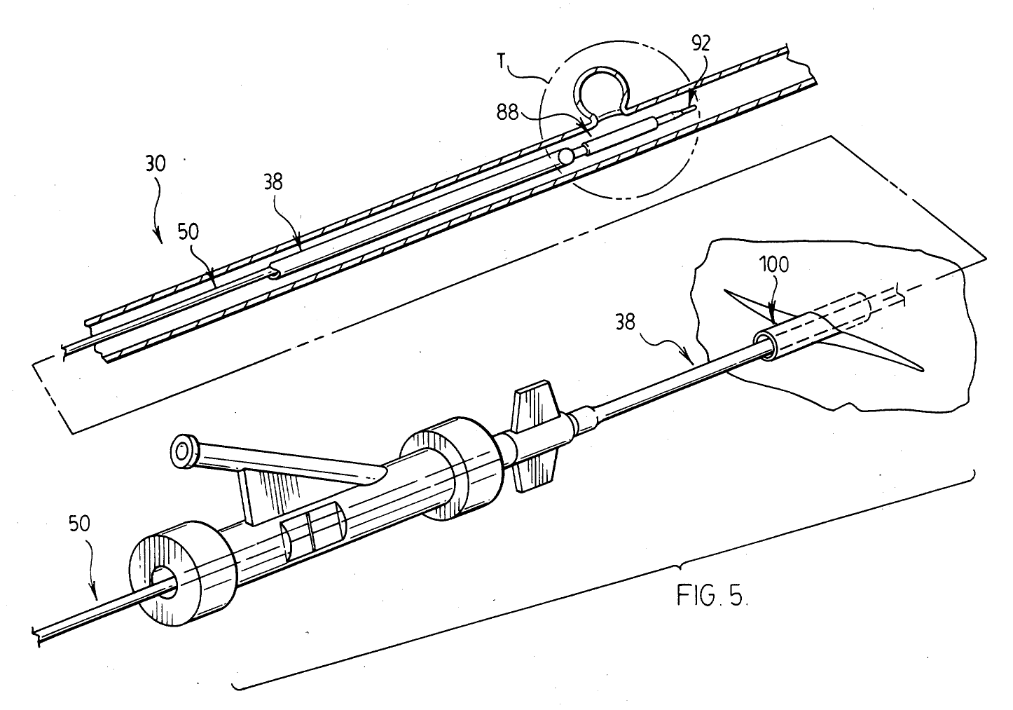

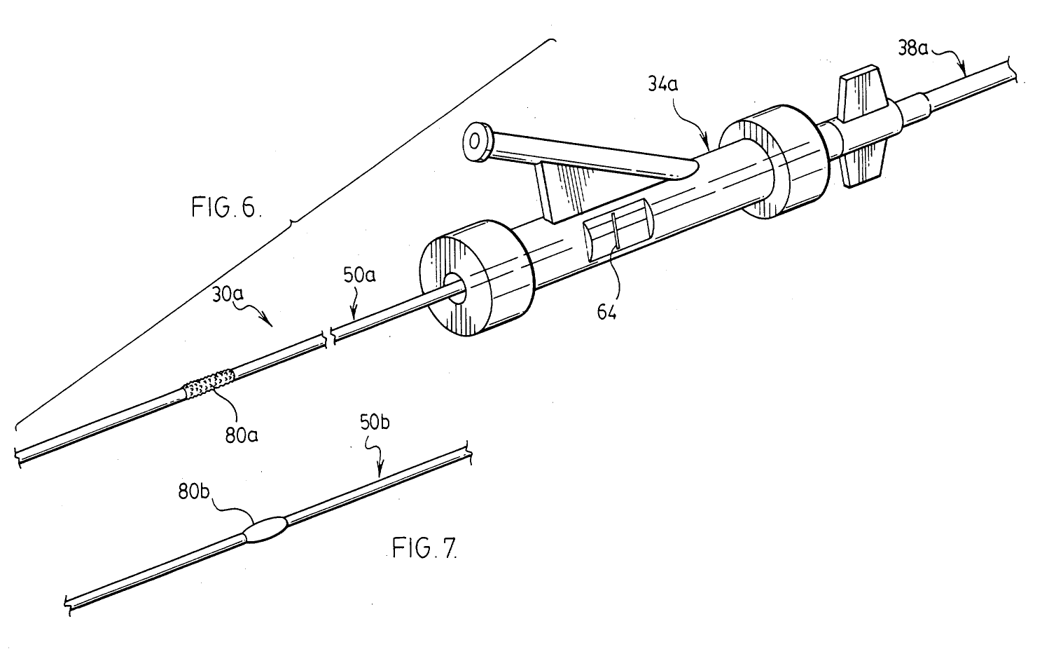

It is to be understood that in other embodiments of the invention graduation 80 could also be other types of locating means or indicia that relies on different sensory perceptions on the part of surgeon S. For example, as shown in FIG. 6, such locating indicium could be a textured surface 80a in lieu of graduation 80 (but could also be used in addition to graduation 80). Textured surface 80a us located on the exterior of microcatheter 50a. Such textured surface could allow surgeon S to use sensory feedback to feel where microcatheter 50a is located in relation to adapter 34a by means of a change in resistence experienced by the surgeon S when inserting the microcatheter. Textured surface 80a could be used in lieu of, or in addition to graduation 80, and thereby obviate the need for gauge 64. Accordingly, while adapter 34a of FIG. 6 includes gauge 64 it is to be understood that gauge 64 can be excluded from the persent embodiment.

Furthermore, other locating means could include a combination of textured surfaces could be applied to the interior of adapter 34a and the exterior of microcatheter 50a, such that surgeon S experiences resistance when that textured surface of microcatheter 50 passes the textured interior of microcatheter 50. Additionally, as shown in, in FIG. 7 a bulge 80b can used in lieu of, or in addition to graduation 80 and/or textured surface 80a. Bulge 80b is slightly smaller in diameter than the proximal end 54b of adapter 34b (not shown in FIG. 7), such that a small amount of resistance is experienced by surgeon S when bulge 80b enters proximal end 54b. Still further types of locating indicium will now occur to those of skill in the art.

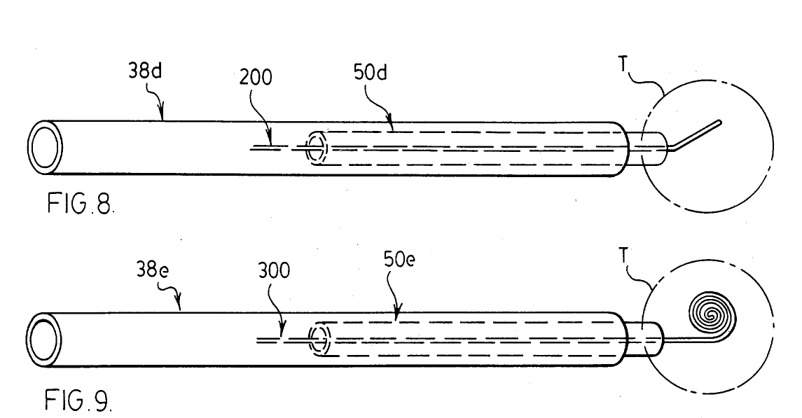

It is to be understood that in other embodiments of the invention, other arrangements of guiding catheters and microcatheters can be constructed. For example, apparatus 30 can be varied such that microcatheter 50 is itself a hollow tube, such that an additional cathether can be inserted within microcatheter 50. The additional catheter would typically include its own graduation or marker at its proximal end that would be located at a position such that when the graduation entered the opening in the proximal end of microcatheter 50, then the additional catheter would be a known distance from exiting tip 92 of the microcatheter 50. By the same token, tip 92 of microcatheter 50 need not have an angioplasty balloon 88, but could include any device for treating a corresponding condition associated with target area T. For example, tip 92 could be characterized by a flexible helical coil, or a bent wire. These variations are shown in FIGS. 8, 9 and 10. As seen in FIGS. 8 there is shown a guiding catheter 38d, which telescopically receives a hollow microcatheter 50d therein, and which in turn telescopically receives a microwire 200. The distal tip of microwire 200 is hockey-stick shaped, but is straightened during travel through microcathther 50d. By the same token, in FIG. 9, there is shown a guiding catheter 38e, which telescopically receives a hollow microcatheter 50e therein, and which in turn telescopically receives a microcoil 300 that has coiled upon its exit from the distal tip of microcatheter 50e.

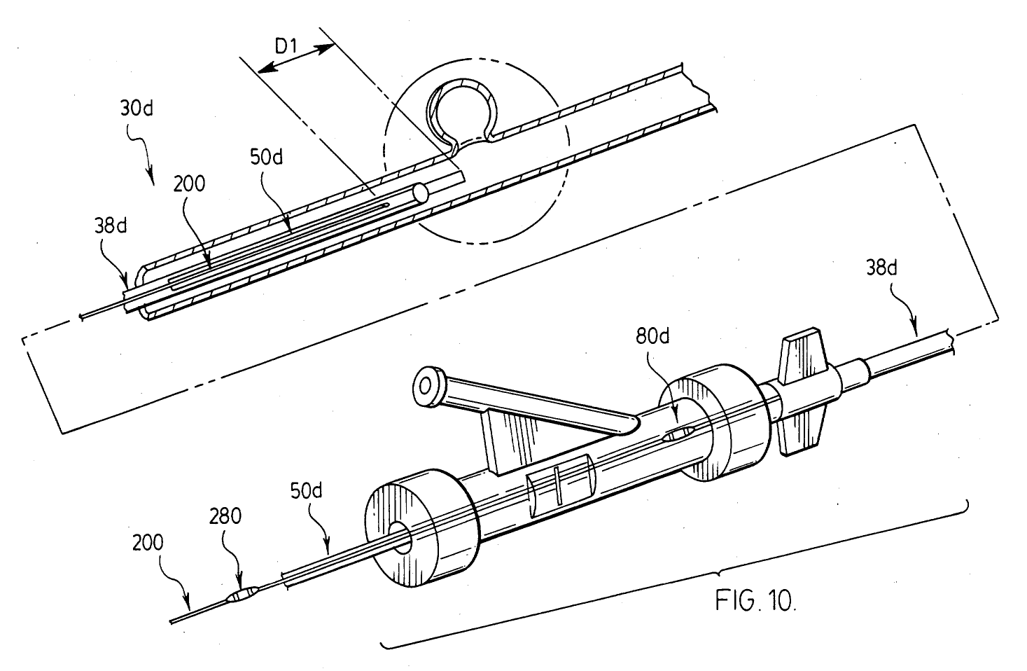

As best seen in FIG. 10, microcathether 50d of FIG. 8 includes a graduation 80d or other indicium to indicate when the distal tip thereof is about to exit from the distal tip of guiding catheter 38d, in substantially the same way graduation 80 on apparatus 30 is configured. However, in addition, the proximal end of microwire 200 also includes a graduation 280, (or other indicium), to indicate to surgeon S when the distal tip of microwire 200 is about to exit the distal tip of its respective microcatheter 50d. In the present embodiment, graduation 280 is located to indicate a given distance D1 (where D1 is indicated on FIG. 10) when graduation 280 is about to enter the proximal end of microcatheter 50d. Graduation 280 is used to indicate when the distal tip of microwire 200 is of a given distance D1 (where D1 is indicated on FIG. 10) of about five mm from exiting the distal tip of its respective microcathether 50d or 50e. Graduation 280 can also be placed to indicate a distance D1 of about seven millimeters, or about ten millimeters, or about fifteen millimeters, as desired. In will now be apparent that configuration in FIG. 10 can be also applied to microcoil 300 of FIG. 9.

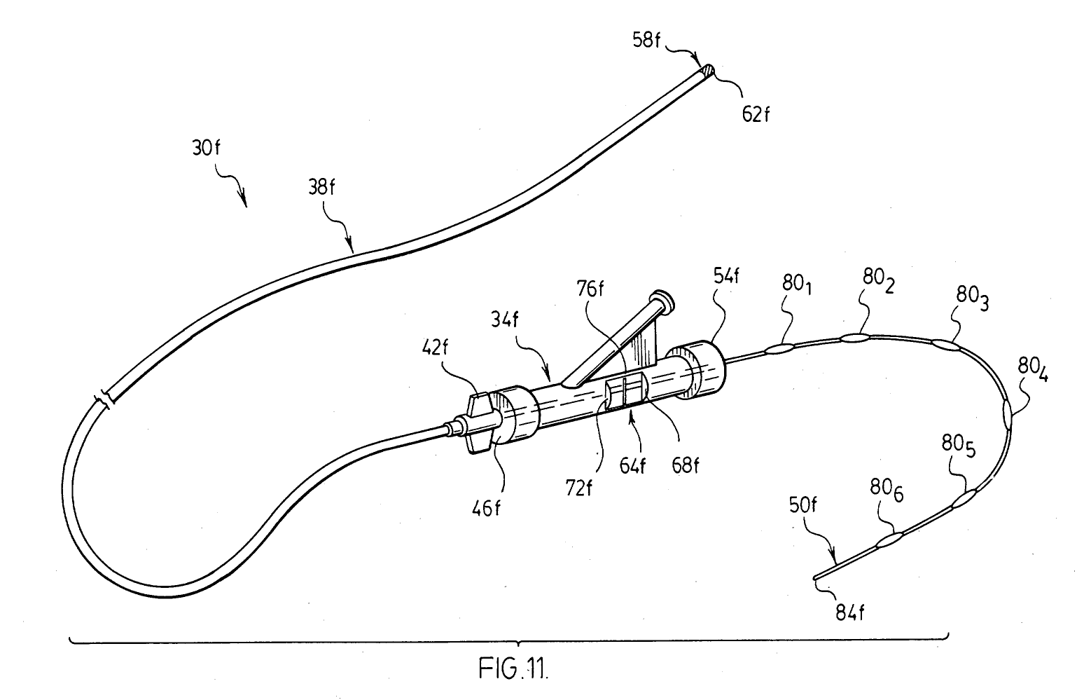

Referring now to FIG. 11, an apparatus for reducing exposure to an imaging beam in accordance with another embodiment of the invention is indicated generally at 30f. Like components in apparatus 30f to components of apparatus 30 in FIG. 1 have the same reference numeral, but followed by the suffix “f”. Thus, apparatus 30f is substantially the same as apparatus 30, except that apparatus 30f includes a plurality of graduations indicated by reference numerals 80₁, 80₂, 80₃, 80₄, 80₅, 80₆. Graduations 80₁, 80₂, 80₃, 80₄, 80₅, 80₆ are placed along the length of microcatheter 50f at a given distance from the distal tip 92f of microcatheter 50f, and accordingly, when inserted into a guiding catheter 38f, each graduation 80₁, 80₂, 80₃, 80₄, 80₅, 80₆ will indicate a different distance that microcatheter 50f has been inserted within guiding catheter 38f. Table I shows a list of presently preferred locations for each graduation 80₁, 80₂, 80₃, 80₄, 80₅, 80₆ and the distances that are represented thereby. (Note that FIG. 11 is not drawn to scale.)

TABLE I

| OVERALL | DISTANCE OF GRADUATION | | | | | |
|---|---|---|---|---|---|---|
| LENGTH IN CM | FROM DISTAL TIP 92F OF | | | | | |
| OF | MICROCATHETER 50F | | | | | |
| MICROCATHETER | GRADUATION | | | | | |
| 50F | 80₁ | 80₂ | 80₃ | 80₄ | 80₅ | 80₆ |
| 155 | 15 | 30 | 60 | 90 | 120 | 150 |
| 125 | 20 | 40 | 60 | 80 | 100 | 120 |
| 105 | 5 | 20 | 40 | 60 | 80 | 100 |

From examining Table I, it will now be apparent that microcathethers 50f of different lengths can be interchangeably used with a plurality of different guiding catheters 38f each having different lengths, and that graduations 80₁, 80₂, 80₃, 80₄, 80₅, 80₆ can thus be used to allow an operator to know how far a particular microcatheter 50f has been inserted into a particular guiding catheter 38f, and thereby derive the approximate distance of how far the distal tip of that particular microcatheter 50f is from exiting the distal tip of that particular guiding catheter 38f. For example, assume that a microcatheter 50f of an overall length of one-hundred-and-fifty-five centimeters marked with graduations 80₁, 80₂, 80₃, 80₄, 80₅, 80₆ as shown in Table I is being used. Further assume that a guiding catheter 38f coupled with adapter 34f has an overall length of one-hundred-and-forty centimeters. Further assume that central indicator 76f of the guiding catheter 38f is located five centimeters from the proximal opening 54f of adapter 34f. Table II shows the relative distance between the distal tip 92f of microcatheter 50f to the point of exiting the distal tip 62f of guiding catheter 38f as each graduation 80₁, 80₂, 80₃, 80₄, 80₅, 80₆ lines up with respective central indicator 76f.

TABLE II

| 155 CM | | | | | | |
|---|---|---|---|---|---|---|
| MICROCATHETER | | | | | | |
| 50F | | | | | | |
| FROM | GRADUATION | | | | | |
| TABLE I | 80₁ | 80₂ | 80₃ | 80₄ | 80₅ | 80₆ |
| DISTANCE IN CM | 130 | 105 | 75 | 45 | 15 | −15 |
| OF DISTAL TIP | | | | | | (MICROCATHER |
| 92F OF | | | | | | TIP |
| MICROCATHETER | | | | | | IS 15 CM |
| 50F FROM | | | | | | PAST |
| EXITING DISTAL | | | | | | GUIDING |
| DIP 62F OF | | | | | | CATHETER |
| GUIDING | | | | | | TIP) |
| CATHETER 38F | | | | | | |
| IN CM THAT | | | | | | |
| MICROCATHETER | | | | | | |
| 50F HAS BEEN | | | | | | |
| INSERTED INTO | | | | | | |
| GUIDING | | | | | | |
| CATHETER 38F | | | | | | |

It will thus now be apparent that microcatheter 50f can be used with guiding catheters 38f of different lengths. It will also be now apparent that multiple graduations can be used on microcoils and/or microwires that run through guiding catheter 38f.

It will now be apparent that any number of graduations can be used, fewer or greater than the six discussed above in Table I, as desired.

While only specific combinations of the various features and components of the present invention have been discussed herein, it will be apparent to those of skill in the art that desired subsets of the disclosed features and components and/or alternative combinations of these features and components can be utilized, as desired. For example, in the embodiment shown in FIGS. 1-5, guiding catheter 38 could be varied to include a graduation or marker of its own located near connector 42 which would represent an approximate length that guiding catheter 38 had been inserted within blood vessel B.

It is also to be understood that other types of locating indicia can be used, other than the specific graduation 80 of microcatheter 50 coupled with gauge 64 of adapter 34. For example gauge 64 can be eliminated altogether, and graduation 80 can be located further towards the proximal end of microcatheter 50, such that the position of tip 92 as shown in FIG. 4 is reflected by the point at which graduation 80 actually enters proximal end 54 of adapter 54.

Furthermore, graduation 80 is described herein as simply being a visual identifying mark located on the shaft of microcatheter 50 that distinguishes that portion of microcatheter 50 from the remainder of microcatheter 50. However, such an identifying mark could be made in colour to make it easier to view.

The embodiments discussed herein refer to having a single graduation 80 along microcatheter 50, however, it is to be understood that a plurality of graduations 80 could be used along the length of microcatheter 50 to represent different positions of tip 92 in relation to its exit from tip 62 of guiding catheter 38.

The above-described embodiments of the invention are intended to be examples of the present invention and alterations and modifications may be effected thereto, by those of skill in the art, without departing from the scope of the invention which is defined solely by the claims appended hereto.

1. An apparatus for reducing exposure to an imaging beam comprising:
a guiding catheter having a distal tip for insertion to a target area and a proximal tip;
a first microcatheter for insertion into said guiding catheter's proximal tip, said first microcatheter having a distal tip that includes a device for treating a condition corresponding to said target area;
a locating means operably associated with at least one of said guiding catheter and said first microcatheter for indicating when said distal tip of said microcathether is about exit said distal tip of said guiding catheter at a predetermined point during said insertion.

2. The apparatus of claim 1 wherein said first microcatheter is hollow and said device is a second microcatheter for insertion into a proximal tip of said first microcatheter and operable to exit said first microcathether's distal tip, said apparatus further including a second locating means operably associated with said second microcatheter for indicating when said second microcathether is about to exit said distal tip of said first microcathether during insertion of said second microcatheter.

3. The apparatus according to claim 2 wherein said second microcatheter is selected from the group consisting of a microwire and a microcoil.

4. The apparatus according to claim 1 wherein said device is a stent.

5. The apparatus according to claim 1 wherein said locating means is a graduation disposed on said first microcatheter that lines up with a predetermined location on said guiding catheter to provide a visual indication when said distal tip of said microcatheter is about exit said distal tip of said guiding catheter.

6. The apparatus according to claim 5 wherein said apparatus further includes a Touhy-Borst adapter releasably-connectable to said guiding catheter and said predetermined location is a proximal open end of said adapter.

7. The apparatus according to claim 5 wherein said apparatus further includes a Touhy-Borst adapter releasably-connectable to said guiding catheter, said adapter is made from a substantially transparent material to allow viewing of said microcatheter passing therethrough, and said predetermined location is an indicator mark located on said adapter.

8. The apparatus according to claim 1 wherein said locating means is a textured surfaces located on said first microcatheter that lines up with a predetermined location on said guiding catheter to provide a tactile indication of when said distal tip of said microcatheter is about exit said distal tip of said guiding catheter.

9. The apparatus according to claim 1 wherein said said apparatus further includes a Touhy-Borst adapter releasably-connectable to said guiding catheter, and said locating means is bulge located on said first microcatheter that has an exterior diameter slightly smaller than an interior diameter of said guiding cathether, such that a resistance to insertion is experienced when said bulge enters a proximal end of said adapter to indicate when said distal tip of said microcatheter is about exit said distal tip of said guiding catheter.

10. The apparatus according to claim 1 wherein said locating means provides said indication when a distal tip of said microcatheter is at a distance of between about zero millimeters to about thirty millimeters from exiting said distal tip of said guiding catheter.

11. The apparatus according to claim 10 wherein said distance is between about two millimeters to about fifteen millimeters.

12. The apparatus according to claim 10 wherein said distance is between about five millimeters to about ten millimeters.

13. The apparatus according to claim 10 wherein said distance is about seven millimeters.

14. An apparatus for reducing exposure to an imaging beam comprising:
a guiding catheter having a distal tip for insertion to a target area and a proximal tip;
an adapter releasably connectable to said guiding catheter, said adapter having a proximal opening at a first end and a connection means at the opposite end, said connection means for connection to said guiding catheter's proximal tip, said adapter being made from a clear material and including a gauge;
a first microcatheter for insertion into said proximal opening and through said guiding catheter, said first microcatheter having a distal tip that includes a device for treating a condition corresponding to said target area when said device exits said guiding catheter's distal tip;
said first microcatheter further including a graduation located towards a proximal end of said first microcatheter, such that when said graduation is lined-up with said gauge said distal tip of said microcatheter is at a predefined distance from exiting said guiding catheter's distal tip.

15. The apparatus according to claim 14 wherein said distance is between about zero millimeters to about thirty millimeters.

16. The apparatus according to claim 14 wherein said distance is between about two millimeters to about fifteen millimeters.

17. The apparatus according to claim 14 wherein said distance is between about five millimeters to about ten millimeters.

18. The apparatus according to claim 14 wherein said distance is about seven millimeters.

19. The apparatus according to claim 14 wherein said target area is a predetermined location inside a patient and said- guiding catheter is inserted into either one of a vein, an artery or a vertebral body of said patient.

20. A method of inserting a microcatheter into a patient comprising:
inserting a guiding catheter into an incision in a patient's skin and passing said guiding catheter through one or more blood vessels until a distal tip of said guiding catheter reaches a target area within said patient; and,
inserting a microcatheter into a proximal opening of said guiding catheter until a locating means located on one of said microcatheter and said guiding catheter indicates that a distal tip of said microcather is at a predetermined distance from exiting a distal tip of said guiding catheter;
exposing said target area to an imaging beam to determine a location of said distal tip of said microcatheter in relation to said target area;
directing said distal tip of said microcatheter towards said target area using said imaging beam for guidance; and,
repeating, as necessary, said exposing step and said directing step until said distal tip of said microcatheter is in a desired location in relation to said target area.
